(19) United States
(12) Patent Application Publication
(10) Pub. No.: US 2008/0221697 A1
(43) Pub. Date: Sep. 11, 2008

(54) Hemi-implant for first metatarsophalangeal joint

(76) Inventor: Robert Graser, San Antonio, TX (US)

Correspondence Address: KAMMER BROWNING PLLC, 7700 BROADWAY, SUITE 202, SAN ANTONIO, TX, 78209, US

(21) Appl. No.: 11/714,993

(22) Filed: Mar. 6, 2007

(51) Int. Cl. A61F 2/42

(52) U.S. Cl. 623/21.19

(57) ABSTRACT

An improved implant for use primarily within the first metatarsophalangeal joint. The implant includes an elliptical, concave, joint surface positioned on a stem and is designed to be placed into a prepared proximal face of the phalanx. The implant is sized according to the patient and may be provided in a number of standard incremental sizes. The improvements relate primarily to the structure, size, and position of the stem that extends into the phalanx to support the implant. The stem is off-set from a center axis of the elliptical joint surface. This off-center placement allows for a longer stem. Single deep indentations on each side of the stem facilitate retention within the bone. A size selection tool is provided to facilitate the selection and placement of an appropriately sized implant. The tool further provides a template for positioning and placing the stem in the phalanx. A method for sizing and selecting the appropriate implant, and positioning and fixing the implant as a partial joint replacement, especially for the first metatarsophalangeal joint, is also described.

### BACKGROUND OF THE INVENTION

1. Field of the Invention

The present invention relates generally to prosthetic medical implant devices, especially those associated with surgical joint replacement. The present invention relates more specifically a partial joint replacement implant for the bones of the human foot, especially the first metatarsophalangeal joints.

2. Description of the Related Art

Prosthetic implant devices have been used for some time to fully or partially replace existing skeletal joints in humans. Among the many joints associated with the bones of the human foot, one that is known to cause frequent problems is the metatarsophalangeal joint between the first metatarsal and the first phalanx in what is commonly known as the hallux or great toe. A number of efforts have been made in the past to partially or fully replace this joint. Some efforts have focused on the partial replacement of the joint using silicone based materials to construct a prosthetic device that is attached to the phalanx (where, for example, the phalanx cartilage has degenerated) and which operates against the metatarsal head which may still remain intact. Silicone material, however, has generally been found to be too soft for the purpose of maintaining an appropriate joint surface and will eventually break down into particles that can have damaging effects on the human body.

Other efforts have been made in the past to fully replace the joint with a unitary flexible silicone implant in such a manner that does not result in an abrasive motion of the remaining bone against the silicone material (as in the case of the silicone hemi-implant). In these cases, the implant actually forms a hinge between the metatarsal and the phalanx and is attached to the resected or planed faces of the metatarsal and the phalanx in cases where both joint surfaces have degraded. Despite the absence of an abrasive motion of the silicone material against a bone surface in the joint, degradation of the silicone material formed into this hinge type joint replacement continues to cause concern for its potential damaging effects on the body. Such full joint replacement using silicone material, therefore, is also not a preferred solution even where both surfaces of the joint require replacement.

A number of metal implant devices, usually made of titanium or its alloys, have been used in place of the above described silicone devices. Full or partial (hemi) replacement of the joint has been provided for in the many efforts to design metal implants for this purpose. The preferred approach where the metatarsal head generally remains intact is to provide for a metal implant device fixed to the phalanx head of the joint. In general, it is preferred not to replace both sides of the joint with metal implants, as this will frequently result in joint discomfort and/or progressive dislocation of the joint often resulting in joint stiffness.

For the above reasons, a hemi-joint replacement is the preferred surgical procedure when the proximal phalanx in the metatarsophalangeal joint has deteriorated while the metatarsal head remains generally sound. Such a replacement is often indicated where the individual has painful arthritis in the joint, painful hallux valgus, hallux limitus, or hallux rigidus.

A number of efforts have been made in the past to provide different types of toe implants for the purpose of partially replacing a metatarsophalangeal joint. An example of a hemi-implant used for replacing the proximal phalanx in the hallux is disclosed in U.S. Pat. No. 5,326,366 issued on Jul. 5, 1994 to Pascarella et al., entitled Biomechanical Great Toe Implant. The device described incorporates a concave surface that imitates the shape of the head of the metatarsal to provide a round or elliptical perimeter to the bearing surface for the joint.

Complete joint replacement has been addressed in a number of other existing patents describing a range of implant designs including: U.S. Pat. No. 5,458,648 issued on Oct. 17, 1995 to Berman et al., entitled Great Toe Joint Implant and Method of Implantation; U.S. Pat. No. 5,314,486 issued on May 24, 1994 to Zang et al., entitled Non-Constrained Total Joint System; U.S. Pat. No. 5,037,440 issued on Aug. 6, 1991 to Koenig, entitled Orthopedic Toe Implant; U.S. Pat. No. 4,903,031 issued on Mar. 13, 1990 to Frisch, entitled Toe Implant; U.S. Pat. No. 6,699,292 issued on Mar. 2, 2004 to Ogilvie et al., entitled Interphalangeal Joint Replacement; U.S. Pat. No. 5,776,203 issued on Jul. 7, 1998 to Spalding et al., entitled Metatarsal Phalangeal Sesamoid Prosthetic Joint; U.S. Pat. No. 4,642,122 issued on Feb. 10, 1987 to Steffee, entitled Toe Implant; U.S. Pat. No. 4,156,296 issued on May 29, 1979 to Johnson et al., entitled Great (Large) Toe Prosthesis and Method of Implanting; and U.S. Pat. No. 6,319,284 B1 issued on Nov. 20, 2001 to Rushdy et al., entitled Toe Implant.

Most of the efforts in the past that have included an implant component designed to be integrated into the phalanx, and more specifically the proximal surface of the phalanx, incorporate a concave bearing surface that is generally in the shape of the distal face of the metatarsal. This provides a generally circular or elliptical perimeter to the bearing surface which helps to maintain the joint in alignment with the implant.

Various efforts have been made to design implant stems that reduce the likelihood of inadequate or improper placement of the implant into the phalanx bone. Many such efforts have created circular or conical stems that, while presenting increased surface area for contact, frequently suffer from rotation after placement. Other efforts at creating suitable stem configurations for the implant have focused on a variety of non-rotating configurations, often at the cost of decreased surface contact. Some of the more effective designs present rectangular shafts that offer serrations and sharp edges to facilitate placement and insertion of the implant. Unfortunately, the adequate use of such rectangular wedge shaped stem designs depends greatly on the condition of the phalanx bone and the ability of the physician to provide a receptor opening in the bone that is neither too large nor too small. Excavating or broaching too much of the bone, of course, provides a loose placement of the implant, while failing to excavate or broach enough of the bone can result in fractures during placement.

Most efforts in the past to design optimally shaped and sized stems for joint replacement implants have, for a number of reasons, centered the stem on the back face of the concave joint replacement structure. Such centered placement, however, fails to recognize that the largest, most stable portion of the phalanx bone available for use as a means for retaining the implant does not lie immediately distal to the center axis of the joint. Rather, as can be seen in the side views associated with the typical human foot skeletal structure, the major portion of the bone available to receive and support the implant is offset above a center line of the joint. Placing the stem of the implant device along the center line of the joint significantly limits the quantity (and often quality) of bone surrounding the stem and further limits how wide the stem can be and how long the stem can extend into the phalanx bone.

### SUMMARY OF THE INVENTION

Addressing the above concerns, and in fulfillment of the above stated objects, the present invention provides an improved hemi-implant for use primarily in conjunction with the first metatarsophalangeal joint. The hemi-implant is constructed with an elliptical, concave, joint surface component that is positioned on a stem component, and is designed to be placed into a prepared proximal face of the first phalanx bone. The implant is sized according to the requirements of the patient and may be provided in a number of incremental standardized implant sizes. The improvements of the present invention relate primarily to the structure, size, and position of the stem that is designed to extend into the phalanx bone to support the joint surface component of the implant. The stem component of the present invention is positioned off-set from a center line axis of the elliptical joint surface component of the implant. The off-center placement of the stem allows for an extended length to the stem, even for the smallest embodiments of the design. A simpler but more effective retention structure is provided on the acute angle edges of the stem. An implant size selection tool is provided for use by the physician as a means to select and place an appropriately sized implant. The size selection tool further provides a template for positioning and placing the stem through the use of a broach tool directed into the planed proximal surface of the phalanx bone. A method for sizing and selecting the appropriate implant; as well as positioning, placing, and fixing the implant as a partial joint replacement, especially for the first metatarsophalangeal joint, is also described.

Structures, geometries, and methods of use associated with the present invention allow the physician to more securely place the implant with less risk of bone damage and further to provide greater comfort to the patient as a result of more accurate placement and more appropriate sizing of the implant. Further benefits of the structural and functional design of the hemi-implant of the present invention will become apparent to those skilled in the art upon an understanding of the detailed description of the preferred embodiments which refers specifically to the drawing figures attached, a brief description of which follows immediately below.

### BRIEF DESCRIPTION OF THE DRAWINGS

FIG. 1 is a detailed side view (dorsal or plantar) of the implant device of the present invention.

FIG. 2 is a second detailed side view (medial or lateral) orthogonal to the view shown in FIG. 1 of the implant device of the present invention.

FIG. 3 is a top plan view showing the front (proximal) joint surface of the implant device of the present invention.

FIG. 4 is a reverse plan view of the back (distal) side of the implant device of the present invention.

FIG. 5 is a side (medial) view of the typical skeletal structure of the human foot showing the implant device of the present invention in place in the first metatarsophalangeal joint.

FIG. 6 is a top (dorsal) view of the skeletal structure of the human foot showing the implant device of the present invention in place in the first metatarsophalangeal joint.

FIG. 7 is a top plan view of a size selection tool appropriate for use in conjunction with the surgical placement of the implant device of the present invention.

FIG. 8 is a flowchart of the method steps associated with surgically placing the implant device of the present invention using the size selection tool shown in FIG. 7.

### DETAILED DESCRIPTION OF THE PREFERRED EMBODIMENTS

Reference is made first to FIG. 1 for a detailed description of the overall structure of the implant device of the present invention. As indicated above, specific structural features of the implant device of the present invention provide unique and beneficial characteristics to the device that facilitate the surgical placement of the device as well as the post-operative comfort of the patient. Implant device 10 as shown in FIG. 1 comprises two primary components, joint surface component 12 and stem component 14. Joint surface component 12 is a slightly elliptical, concave, surface platform that forms the new joint face for the prosthesis. Stem component 14 extends from the back face or back plane of joint surface component 12 and provides the mechanism whereby the implant is attached to the phalanx bone.

Joint surface component 12 is generally constructed with a concave profile to provide the contact surface for the first metatarsophalangeal joint. This surface construction may generally be described as comprising joint surface concavity 16 and joint surface rim 18. In general, the shape and size of both joint surface concavity 16 and joint surface rim 18 are determined by the overall size of implant device 10 and may vary according to the individual patient's requirements.

The back face of joint surface component 12 is generally planar in configuration and is intended to contact the plane of resection on the phalanx bone. As indicated above, stem component 14 extends from this back face of joint surface component 12 and may in the preferred embodiment be machined from the same metal solid as the joint surface component. Alternately, stem component 14 may be attached to the joint surface component 12 with any number of rigid metal-to-metal attachment methods known in the art. Stem component 14 generally comprises a wedge having a diamond-shaped cross section that terminates in stem wedge tip 22.

On each of the acute angle edges of stem component 14, a retention indentation 20 is provided to facilitate the adherence of the stem within the bone, especially after post-operative bone growth. This single deep indentation 20 on each edge of stem 10 is preferred over the multiple shallow serrations found in the prior art. The single indentation facilitates the easy insertion of the stem into the bone after a broaching tool (described below) has initially formed a receptive cavity. The deep indentation provides greater adherence within the bone, especially over time as bone growth extends into the indentation and firmly retains the implant 10 in place. The indentations, as shown in FIG. 1, may each preferably be as much as 20%-30% of the width dimension W of the stem 10.

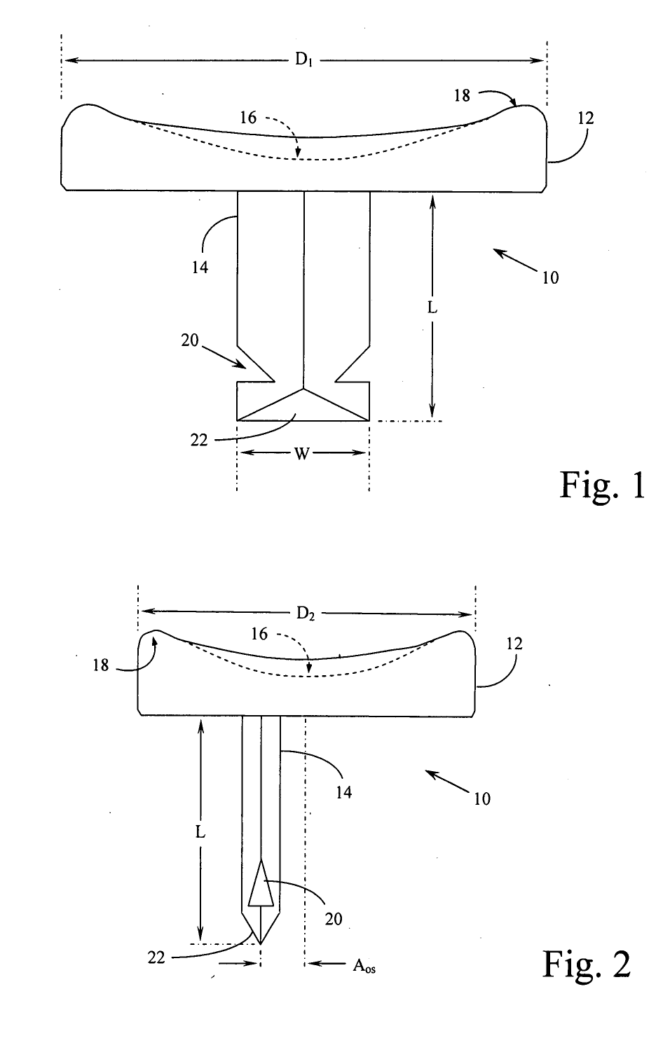

The view shown in FIG. 1 is a side view (dorsal or plantar) of implant device 10 showing the major axis diameter of joint surface component 12. This major axis diameter D₁ is slightly larger than the minor axis diameter D₂ shown in the orthogonal side view of FIG. 2. As indicated above, major axis D₁ (and therefore minor axis diameter D₂) will vary according to the overall size specified for implant device 10. It is anticipated that a range of standard sized implants would be available to the physician to select from, based upon the size of the joint in the particular patient. In general, the major axis diameter D₁ would range from a minimum of approximately 15 mm to a maximum of approximately 25 mm. The most common sizes to be utilized would generally include 17 mm, 19 mm, 21 mm, and 23 mm diameter implants. The features of the present invention, however, are not tied to a specific size of implant and are applicable to implant devices of any diameter within the broad ranges mentioned above.

A primary benefit of the design of the implant device 10 of the present invention is the ability to utilize a stem component 14 of extended length L greater than that typically implemented in the prior art. For reasons discussed in greater detail below, length L of stem component 14 may generally extend 5 mm-10 mm longer than similar implant stems in the prior art. The length L of stem component 14 in the preferred embodiment of the present invention will generally be in the range of 10 mm-40 mm, with a preferred length of approximately 25 mm. Of course the actual length of the stem depends in part on the overall size of the implant with larger implants requiring (and allowing for) larger stem lengths. In this regard it may be practical to view a preferred length dimension L for the stem to be on the order of 5 mm-10 mm longer than the measure of the major axis diameter D₁. Once again, the increased length of dimension L is a result of the off-center placement of stem component 14 as described in more detail below. Width dimension W of stem component 14 is generally related to the overall size of implant device 10 and varies according to the same.

The view shown in FIG. 2 is orthogonal to the view shown in FIG. 1 and, therefore, indicates the minor axis diameter D₂ as discussed above. In this view, joint surface component 12 again is shown to comprise joint surface concavity 16 and joint surface rim 18. The shape and configuration of joint surface component 12 is again formed to generally provide the matching surface for the convex distal face of the metatarsal articular surface. The metatorsalphalangeal joint is generally wider (lateral to medial) than it is deep (dorsal to plantar) therefore providing the differences between major axis diameter D₁ and minor axis diameter D₂.

More importantly, FIG. 2 discloses the positioning of stem component 14 with respect to a central axis of joint surface component. Whereas in the view of FIG. 1 a central axis of stem component 14 is co-axial with the central axis of joint surface component 12, these axes are offset one from the other in the view shown in FIG. 2. This axis offset dimension Aₒₛ is of primary importance in the structural design of the implant device 10 of the present invention. In the preferred embodiment of the present invention, this offset Aₒₛ may be approximately 10%-20% of minor axis diameter D_{2.} As an example, therefore, an implant device having a minor axis diameter of 20 mm may in the preferred embodiment incorporate a stem component axis offset of 2 mm-4 mm. For larger implant device configurations this offset Aₒₛ may be as much as 5 mm-6 mm. In general, however, the offset may best be expressed as a percentage of an average radius of joint surface component 12. Expressed in this manner, stem axis offset Aₒₛ is preferably about 25% of the average radius or approximately ⅛ (D₁+D₂). As indicated above, however, this value may range in the preferred embodiment from as little as 10% to as much as 50% of the average radius of joint surface component 12.

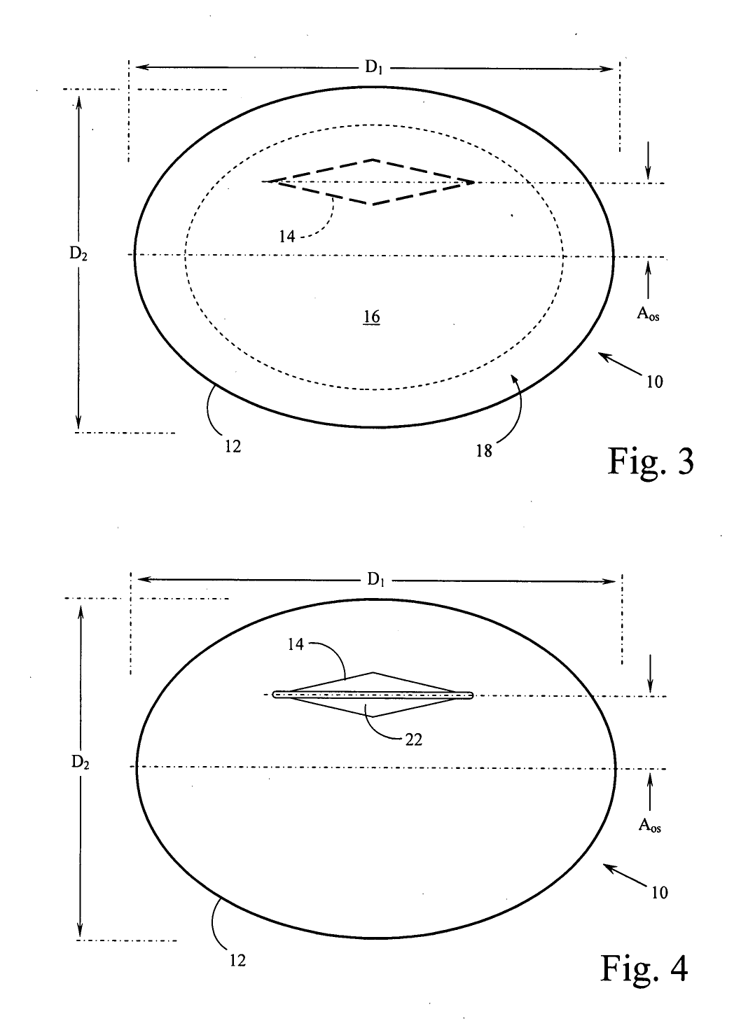

Reference is now made to FIGS. 3 and 4 for a top (proximal) plan view and a bottom (distal) plan view of implant device 10 as described above. In FIG. 3 the generally elliptical shape of joint surface component 12 can be seen. In this view both major axis diameter D₁ and minor axis diameter D₂ are shown. Placement of stem component 14 (shown as a dashed line) can again be seen as displaced across minor axis diameter D₂. The cross-sectional diamond shape of stem component 14 is also disclosed in FIG. 3. Joint surface concavity 16 is shown in FIG. 3 as being generally positioned in a center of the elliptical configuration of joint surface component 12. This configuration facilitates the retention of the implant and its post-operative motion as prosthesis for the joint.

FIG. 4 discloses a distal view of the implant and the placement of stem component 14 and stem wedge tip 22. Once again, stem axis offset Aₒₛ on the base of joint surface component 12 is disclosed.

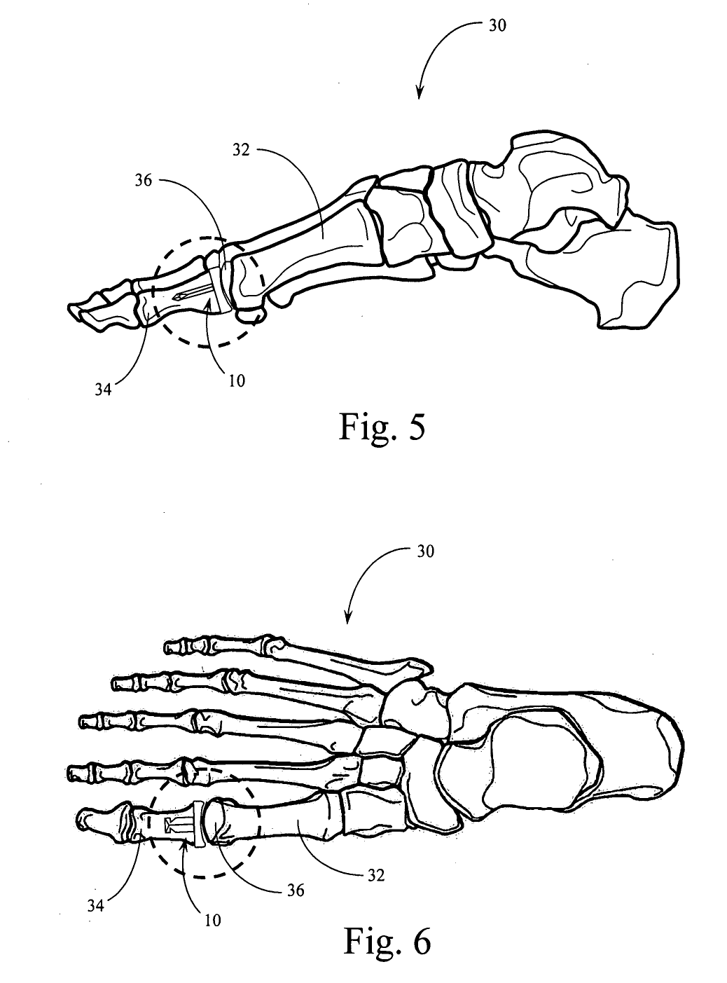

Reference is now made to FIGS. 5 and 6 for a detailed description of the physical placement of implant device 10 and its position in the first metatorsalphalangeal joint of the foot. FIG. 5 is a medial view of the bones of the foot 30 showing the first metatarsal 32 and the first phalanx 34 of the hallux. These bones of the hallux (big toe) together form the first metatorsalphalangeal joint which, as described above, is frequently subject to a variety of abnormal conditions that indicate the replacement of the joint with an implant of the type of the present invention. In the view of FIG. 5, implant device 10 is shown with a portion of the first phalanx 34 cut away to disclose the orientation and placement of the stem of the implant therein.

Of critical note in FIG. 5 is the profile configuration of first phalanx 34 and the manner in which the plantar surface of the bone arches upward in a manner that narrows a mid-section of the bone from a generally wider cross-section at the metatorsalphalangeal (MP) joint and the interphalangeal (IP) joint. The axial offset of the stem of implant device 10 described above effectively centers the stem within the narrower mid-section of the first phalanx bone. This centering provides two advantages to the structural design of the implant device of the present invention. First, it prevent the inadvertent incursion of the stem of implant device 10 through to a point near the plantar face of the first phalanx such that the stem might either actually exit the bone or create a weak point or fracture during placement of the implant. Second, the axial offset of the stem of implant device 10 allows a longer length for the stem extending as it does through the medullary canal region of the first phalanx in a manner that maintains firm support and minimal damage to the bone.

FIG. 6 is a dorsal view of the bones of the foot 30 again showing the placement of implant device 10 within the first phalanx 34 of the foot to form the phalangeal articular surface of the first metatorsalphalangeal joint 36. In this view (and the view shown in FIG. 5) the general convex character of the metatarsal face of the joint can be seen to dictate the general concave structure of the proximal surface of the implant device of the present invention. As seen in FIG. 6 it is apparent that although there is a narrowing in the mid-section of the first phalanx in the medial to lateral direction, such narrowing is symmetrical with respect to a central axis of the bone in contrast to the narrowing in a plantar to dorsal direction shown in FIG. 5. For this reason, it is unnecessary to establish any offset from a center line on the implant device to accommodate this narrowing.

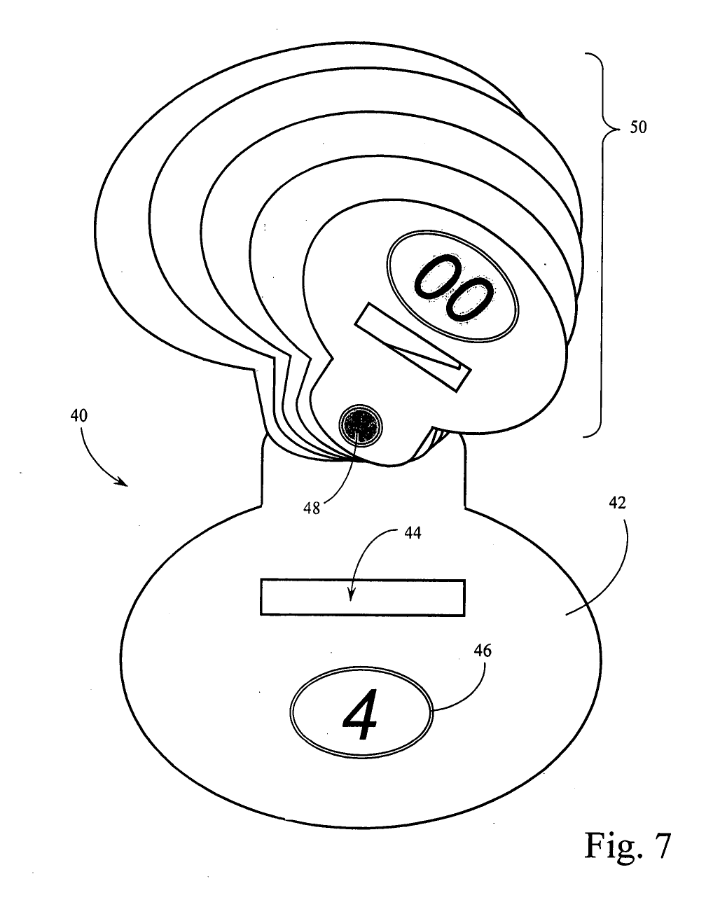

Reference is now made to FIG. 7 for a detailed description of a tool provided to facilitate the selection of an implant device of the appropriate size and the placement of that device on the resected surface of the first phalanx. Sizing template set 40 comprises a plurality of individual templates sized to equal the size and configuration of the joint surface component of the implant device. These generally elliptical templates, therefore, provide the physician with a simple mechanism for measuring the joint prior to the step of selecting an implant. A large sized template 42 is shown in detail in FIG. 7 as comprising a generally elliptical plate having stem placement window 44 and template size indicia 46. The template 42 is attached by way of swivel post 48 to a plurality of additional sized templates 50. One template for each of the variety of implant device sizes would be incorporated onto swivel post 48 in a manner that allows the physician to select one of the templates, position it within the resected joint and determine therefrom the appropriate implant size to be used. The sizing template set 40 shown in FIG. 7 is an example that contains six different sizes although, as indicated above, any number of different sized implant devices may be constructed, varying in size according to the specific needs of a particular patient. Likewise, sizing template set 40 may incorporate any number of individual templates depending upon the variety and number of implant device sizes.

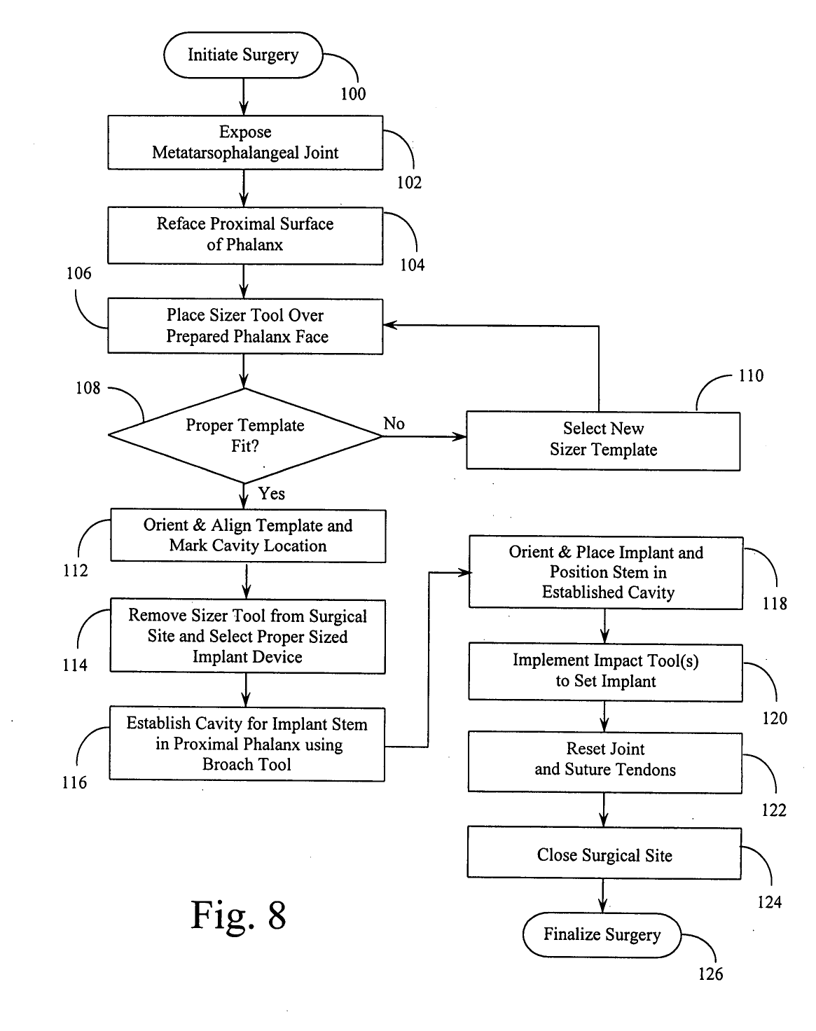

Reference is finally made to FIG. 8 for a brief description of the methodology associated with utilizing the sizing template device of the present invention and the selection and placement of an appropriately sized implant device. It is recognized that the steps described in FIG. 8 are general in nature and do not include many of the individual specific surgical procedure steps that would be required for the type of prosthetic placement associated with a hemiarthroplasty resurfacing procedure. The steps shown in FIG. 8, however, do disclose those aspects of the method that allow the physician to improve the placement of the implant device. The surgery is initiated at Step 100, with the process of exposing the metatorsalphalangeal joint at Step 102. An appropriate portion of the proximal surface of the first phalanx is resected at Step 104. The physician may then utilize the sizer tool by placing the tool over the prepared phalanx face and selecting the most appropriate template size to match the prepared face. The indicia provided on each of the template elements in the tool provide the physician with the appropriate implant device size to be utilized.

If the selected template size is not the proper fit as determined at Step 108 the physician selects a new size of template at Step 110 and again places the sizer tool over the prepared phalanx face at Step 106. If at Step 108 a proper template fit is identified, then the physician proceeds at Step 112 to orient and align the template and thereafter mark the cavity location for the stem of the implant. This location is determined by appropriate marking of the articular surface of the phalanx (as resected) through the template window 44 (best seen in FIG. 7). The physician then removes the sizing tool at Step 114 from the surgical site and selects the proper sized implant device that matches the indicia on the selected template tool.

At Step 116 a cavity is established for the implant stem utilizing a broach tool directed in to the marked location on the proximal surface of the phalanx. Once the cavity is established the physician then orients and places the implant device stem within the established cavity at Step 118. At Step 120 an impact tool is implemented to set the implant in place within the phalanx as described and shown above. The physician then proceeds to re-set the joint and suture any tendons that have been displaced in the process of placing the implant at Step 122. The surgical site is closed at Step 124 and the surgery is finalized at Step 126 in a manner will known in the art.

Although the present invention has been described in terms of the foregoing preferred embodiments, this description has been provided by way of explanation only, and is not intended to be construed as a limitation of the invention. Those skilled in the art will recognize modifications of the present invention that might accommodate specific patients and bone or joint structures. As is known in the art, it is necessary to provide various sizes of a similarly structured implant device in order to accommodate patients of different ages and different bone structures. Such modifications as to components, size, and even configuration where such modifications are merely coincidental to the size of the patient, do no necessarily depart from the spirit and scope of the invention. It is further anticipated that some variation may occur, for example, in the configuration of the various sections of the implant device to allow variations in the force required when placing the device. Again, all of these various modifications and variations do not necessarily depart from the spirit and scope of the invention.

I claim:

1. A prosthetic implant for partial replacement of the first metatarsophalangeal joint in the human foot, the implant comprising:
a generally elliptical joint surface component configured to serve as the proximal face of the first phalanx bone of the foot; and
an elongated stem extending distally from the joint surface component, the stem providing an anchor for the implant device within the first phalanx bone, the stem having a longitudinal axis generally orthogonal to a plane formed by the joint face component and off-set from a line orthogonal to a center point of the generally elliptical joint surface component.

2. The implant device of claim 1 wherein the generally elliptical joint surface component comprises a metal material and the elongated stem component comprises a metal material.

3. The implant device of claim 2 wherein the metal material of the joint surface component comprises a metal material selected from a group consisting of: a titanium alloy, a cobalt chrome alloy, and stainless steel.

4. The implant device of claim 1 wherein the generally elliptical joint surface component has a major diameter and a minor diameter and the longitudinal axis of the stem is generally aligned with a mid-point on the major diameter and is generally off-set from a mid-point on the minor diameter.

5. The implant device of claim 4 wherein the off-set of the longitudinal axis of the stem from the mid-point on the minor diameter is a percentage of the minor diameter in the range from 10% of the minor diameter to 20% of the minor diameter.

6. The implant device of claim 4 wherein the off-set of the longitudinal axis of the stem from the mid-point on the minor diameter is a percentage of the minor diameter approximately equal 12.5% of the minor diameter.

7. The implant device of claim 1 wherein the elongated stem component comprises a diamond shaped cross-section having two acute angled edges and two obtuse angled edges.

8. The implant device of claim 7 wherein the two acute angled edges of the elongated stem component each partially define at least one angled indentation into a width dimension of the stem component.

9. The implant device of claim 8 wherein the at least one angled indentation on each of the two acute angled edges of the stem component comprises a single angled indentation on each of the edges, the angled indentations each having a depth into the width dimension of the stem component in the range of 20% to 30% of the width dimension.

10. The implant device of claim 1 wherein the elongated stem component further comprises a terminal wedge at a distal end of the stem component.

11. The implant device of claim 1 wherein the elongated stem component comprises a longitudinal structure extending from a point of attachment on a distal side of the joint surface component to a length generally in the range of 10 mm-40 mm.

12. The implant device of claim 1 wherein the elongated stem component comprises a longitudinal structure extending from a point of attachment on a distal side of the joint surface component to a length of approximately 25 mm.

13. A system for sizing and surgically placing a prosthetic implant for partial replacement of the first metatarsophalangeal joint in the human foot, the system comprising:
a prosthetic implant, the implant comprising:
a generally elliptical joint surface component configured to serve as the proximal face of the first phalanx bone of the foot;
an elongated stem component extending distally from the joint surface component, the stem providing an anchor for the implant device within the first phalanx bone, the stem having a longitudinal axis generally orthogonal to a plane formed by the joint face component and off-set from a line orthogonal to a center point of the generally elliptical joint surface component; and
an implant sizing tool comprising a plurality of sizing templates selectable for temporary placement in proximity to the proximal face of the first phalanx, each sizing template of the implant sizing tool comprising an elliptical plate corresponding in size and shape to one of a number of different sized implants.

14. The system of claim 13 wherein each sizing template of the implant sizing tool further defines a stem positioning window suitable for identifying a proper location on the proximal surface of the phalanx bone for placement of the stem component of the implant.

15. The system of claim 13 wherein each sizing template of the implant sizing tool further comprises indicia for identifying a corresponding implant size to be used when that sizing template is selected and identified as a proper size for the joint replacement.

16. A method for implanting a prosthetic device for partial replacement of the first metatarsophalangeal joint in the human foot, the method comprising the steps of:
surgically exposing the metatarsophalangeal joint;
resecting and refacing a proximal surface of the phalanx;
providing an implant sizing tool, the implant sizing tool comprising a plurality of sizing templates selectable for temporary placement in proximity to the proximal surface of the phalanx, each sizing template of the implant sizing tool comprising an elliptical plate corresponding in size and shape to one of a number of different sized prosthetic implants;
selecting one of the plurality of sizing templates that most closely approximates a proper sized prosthetic implant for the size of the joint;
orienting and aligning the selected sizing template on the proximal surface of the phalanx;
marking a position of a cavity to be formed into the phalanx for anchoring the proper sized implant device;
establishing a cavity into the proximal phalanx using a broach tool;
providing a plurality of different sized implant devices, each of the implants comprising:
a generally elliptical joint surface component configured to serve as the proximal face of the first phalanx bone of the foot;
an elongated stem component extending distally from the joint surface component, the stem providing an anchor for the implant device within the first phalanx bone, the stem having a longitudinal axis generally orthogonal to a plane formed by the joint face component and off-set from a line orthogonal to a center point of the generally elliptical joint surface component;
selecting one of the plurality of different sized implant devices corresponding to the selected one of the plurality of sizing templates;
orienting and positioning the selected implant device on the proximal surface of the phalanx;
positioning the stem of the implant device into the established cavity in the proximal surface of the phalanx;
setting the stem of the implant device into a medullary region of the phalanx using an impact tool; and
closing the surgical site associated with the metatarsophalangeal joint.
